(19) United States
(12) Patent Application Publication
(10) Pub. No.: US 2007/0179615 A1
(43) Pub. Date: Aug. 2, 2007

(54) Intervertebral prosthetic disc

(75) Inventors: Eric S. Heinz, Memphis, TN (US); Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/344,602

(22) Filed: Jan. 31, 2006

(51) Int. Cl. A61F 2/44

(52) U.S. Cl. 623/17.12; 623/17.14

(57) ABSTRACT

An intervertebral prosthetic disc is disclosed and can be installed within an intervertebral space between a first vertebra and a second vertebra. The intervertebral prosthetic disc can include a first component that can have a first compliant structure that can be configure to engage the first vertebra. Further, the first compliant structure can at least partially conform to a shape of the first vertebra. The intervertebral prosthetic disc can also include a second component that can be configured to engage the second vertebra.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics. of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is an anterior view of a first embodiment of an intervertebral prosthetic disc;

FIG. 5 is an exploded anterior view of the first embodiment of the intervertebral prosthetic disc;

FIG. 6 is a lateral view of the first embodiment of the intervertebral prosthetic disc;

FIG. 7 is an exploded lateral view of the first embodiment of the intervertebral prosthetic disc;

FIG. 8 is a plan view of a superior half of the first embodiment of the intervertebral prosthetic disc;

FIG. 9 is another plan view of the superior half of the first embodiment of the intervertebral prosthetic disc;

FIG. 10 is a plan view of an inferior half of the first embodiment of the intervertebral prosthetic disc;

FIG. 11 is a plan view of an inferior half of the first embodiment of the intervertebral prosthetic disc;

FIG. 12 is an exploded lateral view of the first embodiment of the intervertebral prosthetic disc installed within an intervertebral space between a pair of adjacent vertrebrae;

FIG. 13 is an anterior view of the first embodiment of the intervertebral prosthetic disc installed within an intervertebral space between a pair of adjacent vertrebrae;

FIG. 14 is an anterior view of a second embodiment of an intervertebral prosthetic disc;

FIG. 15 is an exploded anterior view of the second embodiment of the intervertebral prosthetic disc;

FIG. 16 is a lateral view of the second embodiment of the intervertebral prosthetic disc;

FIG. 17 is an exploded lateral view of the second embodiment of the intervertebral prosthetic disc;

FIG. 18 is a plan view of a superior half of the second embodiment of the intervertebral prosthetic disc;

FIG. 19 is another plan view of the superior half of the second embodiment of the intervertebral prosthetic disc;

FIG. 20 is a plan view of an inferior half of the second embodiment of the intervertebral prosthetic disc;

FIG. 21 is another plan view of the inferior half of the second embodiment of the intervertebral prosthetic disc;

FIG. 22 is a lateral view of a third embodiment of an intervertebral prosthetic disc;

FIG. 23 is an exploded lateral view of the third embodiment of the intervertebral prosthetic disc;

FIG. 24 is a anterior view of the third embodiment of the intervertebral prosthetic disc;

FIG. 25 is a perspective view of a superior component of the third embodiment of the intervertebral prosthetic disc;

FIG. 26 is a perspective view of an inferior component of the third embodiment of the intervertebral prosthetic disc;

FIG. 27 is a lateral view of a fourth embodiment of an intervertebral prosthetic disc;

FIG. 28 is an exploded lateral view of the fourth embodiment of the intervertebral prosthetic disc;

FIG. 29 is a anterior view of the fourth embodiment of the intervertebral prosthetic disc;

FIG. 30 is a perspective view of a superior component of the fourth embodiment of the intervertebral prosthetic disc; and

FIG. 31 is a perspective view of an inferior component of the fourth embodiment of the intervertebral prosthetic disc.

### DETAILED DESCRIPTION OF THE DRAWINGS

An intervertebral prosthetic disc is disclosed and can be installed within an intervertebral space between a first vertebra and a second vertebra. The intervertebral prosthetic disc can include a first component that can have a first compliant structure that can be configure to engage the first vertebra. Further, the first compliant structure can at least partially conform to a shape of the first vertebra. The intervertebral prosthetic disc can also include a second component that can be configured to engage the second vertebra.

In another embodiment, an intervertebral prosthetic disc is disclosed and can be installed within an intervertebral space between an inferior vertebra and a superior vertebra. The intervertebral prosthetic disc can include an inferior support plate that can have an inferior compliant structure attached thereto. The inferior compliant structure can be configured to conform to the inferior vertebra. Moreover, the intervertebral prosthetic disc can include a superior support plate that can have a superior compliant structure attached thereto. The superior compliant structure can also be configured to conform to the superior vertebra.

In yet another embodiment, an intervertebral prosthetic disc is disclosed and can be installed within an intervertebral space between an inferior vertebra and a superior vertebra. The intervertebral prosthetic disc can include a superior component that can include a superior support plate and a superior compliant structure that can be affixed to the superior bearing surface. Further, the intervertebral prosthetic disc can include an inferior component that can include an inferior support plate and an inferior compliant structure affixed to the inferior bearing surface. Also, the intervertebral prosthetic disc can include a nucleus that can be disposed between the superior component and the inferior component. The nucleus can be configured to allow relative motion between the superior component and the inferior component.

Description of Relevant Anatomy

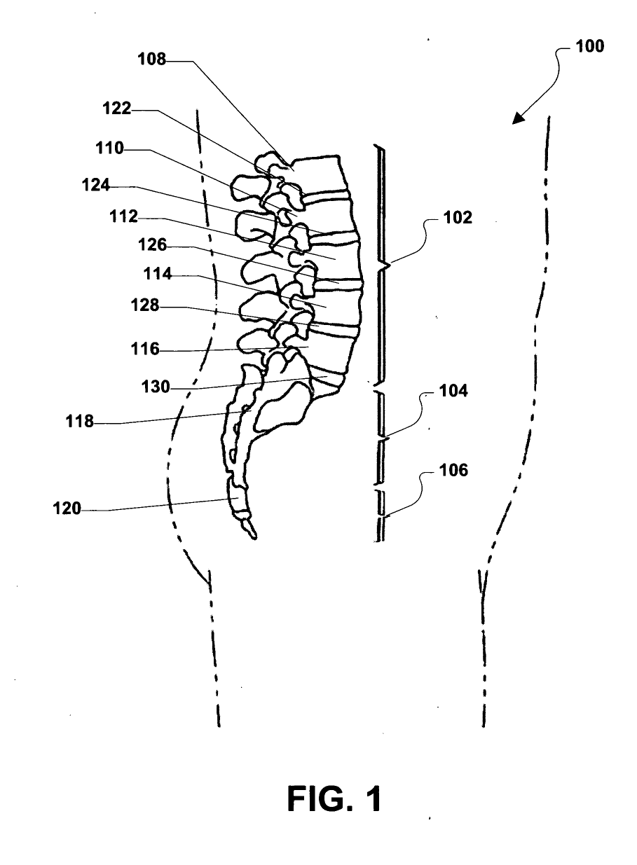

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

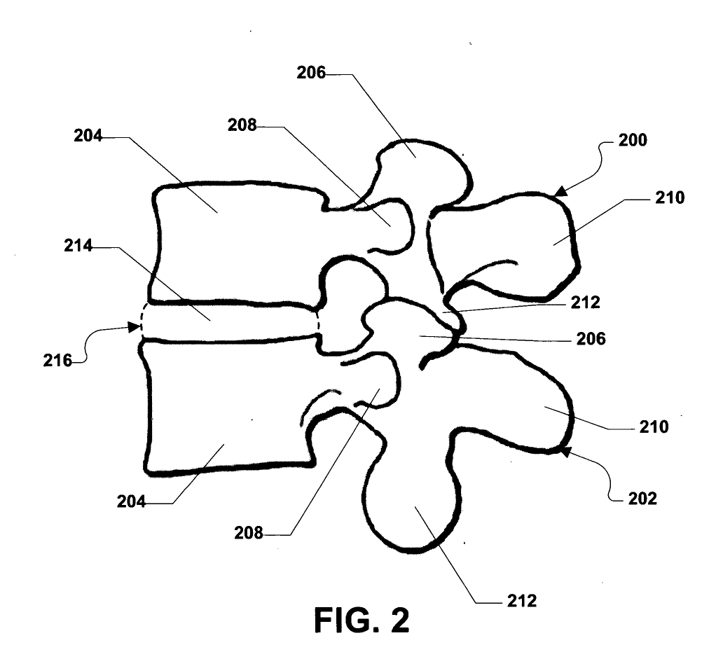

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

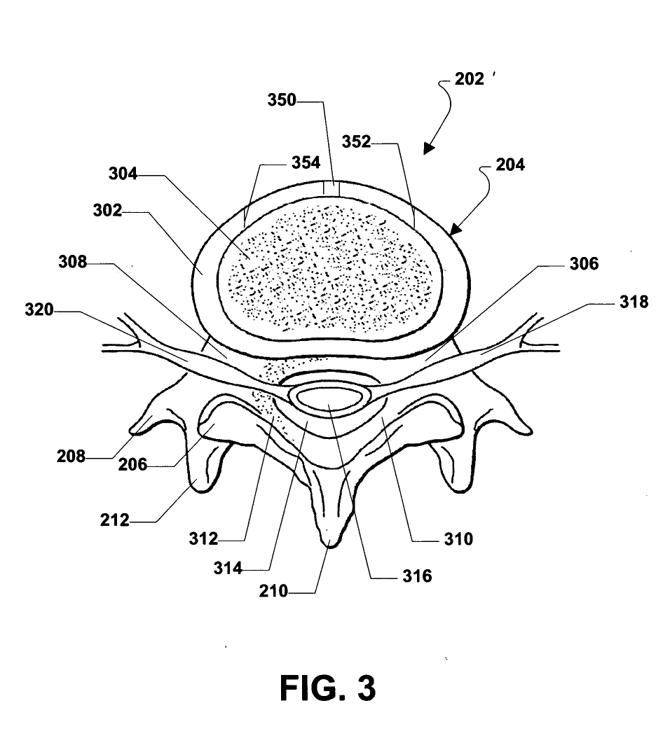

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

Description of a First Embodiment of an Intervertebral Prosthetic Disc

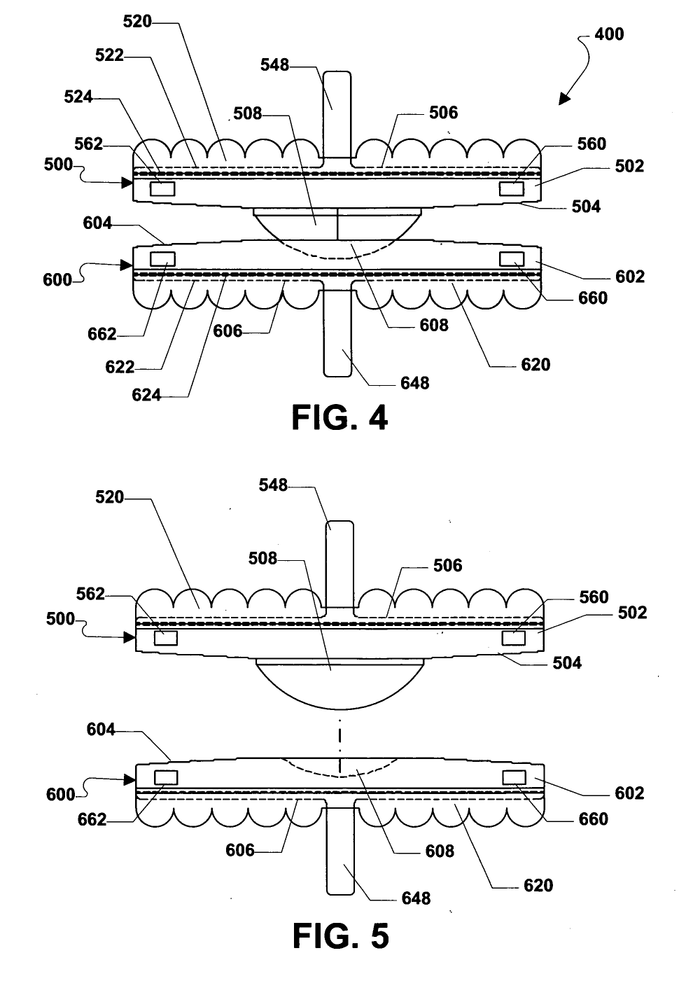

Referring to FIGS. 4 through 11 a first embodiment of an intervertebral prosthetic disc is shown and is generally designated 400. As illustrated, the intervertebral prosthetic disc 400 can include a superior component 500 and an inferior component 600. In a particular embodiment, the components 500, 600 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 500, 600 can be made from any other substantially rigid biocompatible materials.

In a particular embodiment, the superior component 500 can include a superior support plate 502 that has a superior articular surface 504 and a superior bearing surface 506. In a particular embodiment, the superior articular surface 504 can be generally curved and the superior bearing surface 506 can be substantially flat. In an alternative embodiment, the superior articular surface 504 can be substantially flat and at least a portion of the superior bearing surface 506 can be generally curved.

As illustrated in FIG. 4 through FIG. 7, a projection 508 extends from the superior articular surface 504 of the superior support plate 502. In a particular embodiment, the projection 508 has a hemi-spherical shape. Alternatively, the projection 508 can have an elliptical shape, a cylindrical shape, or other arcuate shape. Moreover, the projection 508 can be formed with a groove 510.

As further illustrated, the superior component 500 can include a superior compliant structure 520 that can be affixed, or otherwise attached to the superior component 500. In a particular embodiment, a groove 522 can be formed in the superior component 500, e.g., around the perimeter of the superior component 500. A wire 524 can secure the superior compliant structure 520 within the groove 522. For example, the ends of the wire 524 may be laser welded to each other to create a permanent tension band.

In an alternative embodiment, the superior compliant structure 520 can be chemically bonded to the superior bearing surface 506, e.g., using an adhesive or another chemical bonding agent. Further, the superior compliant structure 520 can be mechanically anchored to the superior bearing surface 506, e.g., using hook-and-loop fasteners, or another type of fastener.

In a particular embodiment, after installation, the superior compliant structure 520 can be in direct contact with vertebral bone, e.g., cortical bone and cancellous bone. Further, in a particular embodiment, the superior compliant structure 520 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the superior compliant structure 520 can be made from a three-dimensional (3-D) woven structure, e.g., a three-dimensional (3-D) polyester structure. Further, in a particular embodiment, the superior compliant structure 520 can be resorbable, non-resorbable, or a combination thereof.

In a particular embodiment, the superior compliant structure 520 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant structure 520 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 4 through FIG. 7 indicate that the superior component 500 can include a superior keel 548 that extends from superior bearing surface 506. During installation, described below, the superior keel 548 can at least partially engage a keel groove that can be established within a cortical rim of a vertebra. Further, the superior keel 548 can be coated with a bone-growth promoting substance, e.g., a hydroxyapatite coating formed of calcium phosphate. Additionally, the superior bearing surface 506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

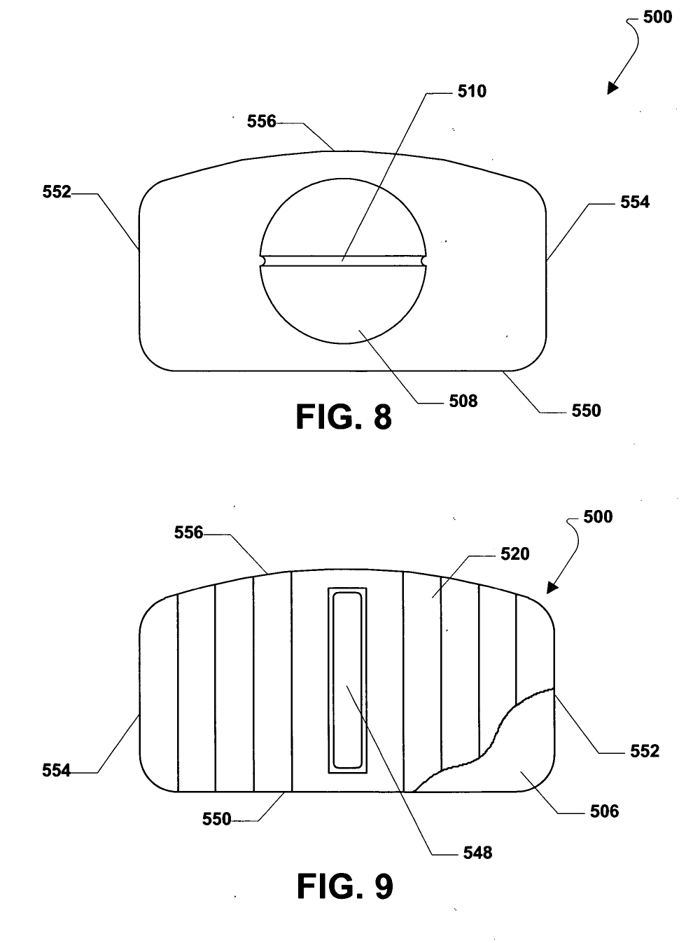

As illustrated in FIG. 8 and FIG. 9, the superior component 500 can be generally rectangular in shape. For example, the superior component 500 can have a substantially straight posterior side 550. A first straight lateral side 552 and a second substantially straight lateral side 554 can extend substantially perpendicular from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

FIG. 4 and FIG. 5 show that the superior component 500 includes a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 11.

In a particular embodiment, the inferior component 600 can include an inferior support plate 602 that has an inferior articular surface 604 and an inferior bearing surface 606. In a particular embodiment, the inferior articular surface 604 can be generally curved and the inferior bearing surface 606 can be substantially flat. In an alternative embodiment, the inferior articular surface 604 can be substantially flat and at least a portion of the inferior bearing surface 606 can be generally curved.

As illustrated in FIG. 4 through FIG. 7, a depression 608 extends into the inferior articular surface 604 of the inferior support plate 602. In a particular embodiment, the depression 608 is sized and shaped to receive the projection 508 of the superior component 500. For example, the depression 608 can have a hemi-spherical shape. Alternatively, the depression 608 can have an elliptical shape, a cylindrical shape, or other arcuate shape.

As further illustrated, the inferior component 600 can include an inferior compliant structure 620 that can be affixed, or otherwise attached to the inferior component 600. In a particular embodiment, a groove 622 can be formed in the inferior component 600, e.g., around the perimeter of the inferior component 600. A wire 624 can secure the inferior compliant structure 620 within the groove 622. For example, the ends of the wire 624 may be laser welded to each other to create a permanent tension band.

In an alternative embodiment, the inferior compliant structure 620 can be chemically bonded to the inferior bearing surface 606, e.g., using an adhesive or another chemical bonding agent. Further, the inferior compliant structure 620 can be mechanically anchored to the inferior bearing surface 606, e.g., using hook-and-loop fasteners, or another type of fastener.

In a particular embodiment, after installation, the inferior compliant structure 620 can be in direct contact with vertebral bone, e.g., cortical bone and cancellous bone. Further, in a particular embodiment, the inferior compliant structure 620 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the inferior compliant structure 620 can be made from a three-dimensional (3-D) woven structure, e.g., a three-dimensional (3-D) polyester structure. Further, in a particular embodiment, the superior compliant structure 620 can be resorbable, non-resorbable, or a combination thereof.

In a particular embodiment, the inferior compliant structure 620 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant structure 620 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 4 through FIG. 7 indicate that the inferior component 600 can include an inferior keel 648 that extends from inferior bearing surface 606. During installation, described below, the inferior keel 648 can at least partially engage a keel groove that can be established within a cortical rim of a vertebra, e.g., the keel groove 70 shown in FIG. 3. Further, the inferior keel 648 can be coated with a bone-growth promoting substance, e.g., a hydroxyapatite coating formed of calcium phosphate. Additionally, the inferior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

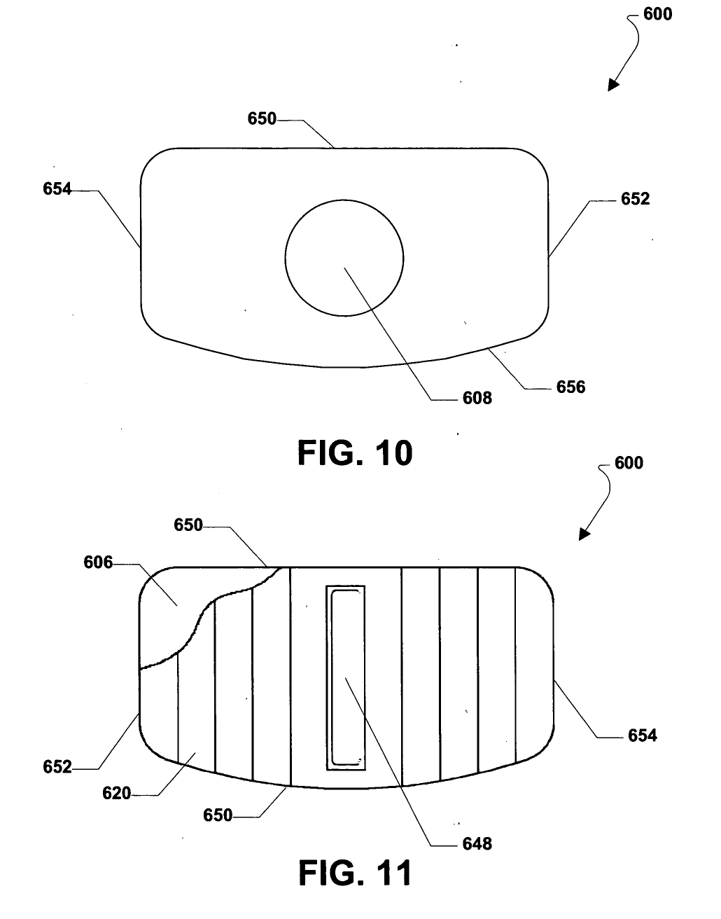

In a particular embodiment, as shown in FIG. 10 and FIG. 11, the inferior component 600 can be shaped to match the shape of the superior component 500, shown in FIG. 8 and FIG. 9. Further, the inferior component 600 can be generally rectangular in shape. For example, the inferior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

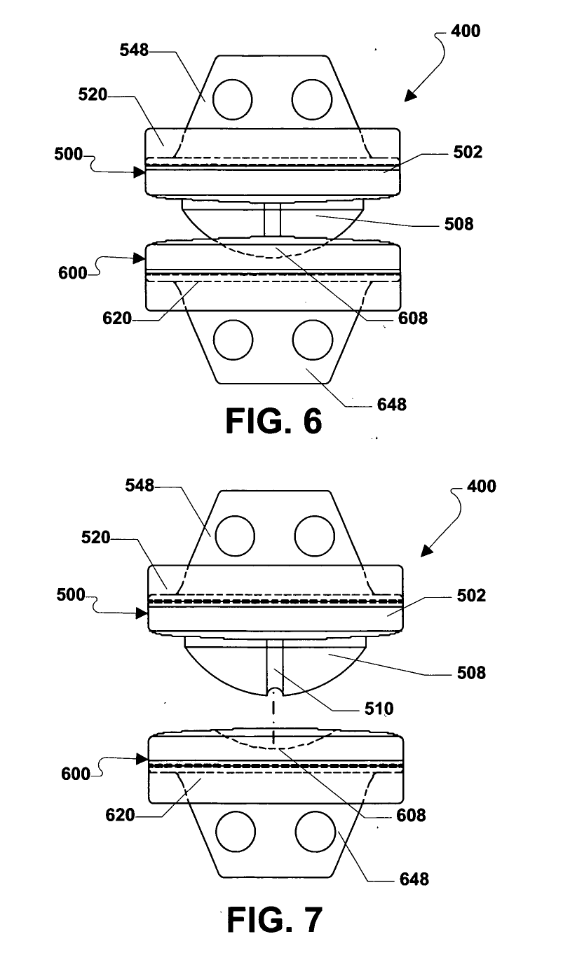

FIG. 4 and FIG. 6 show that the inferior component 600 includes a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 9.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm).

Installation of the First Embodiment within an Intervertebral Space

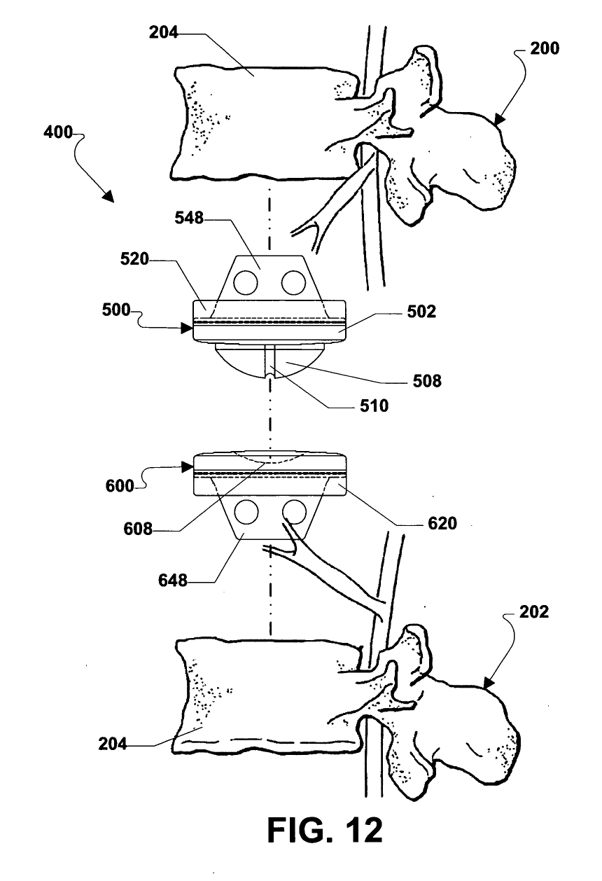

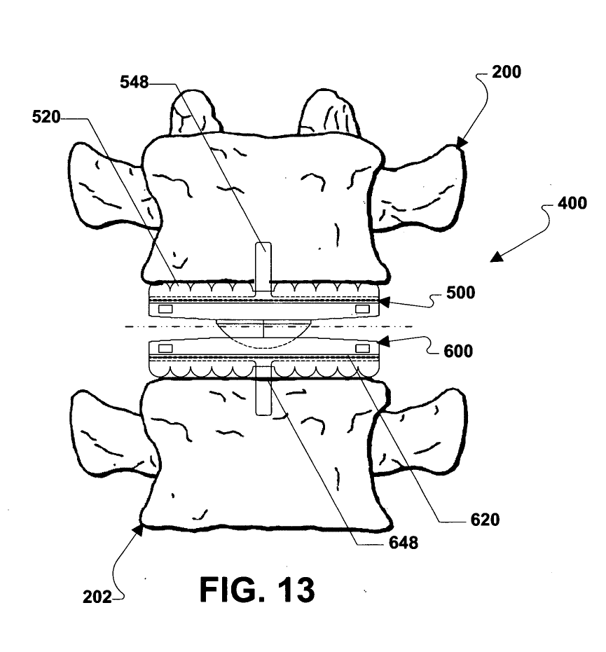

Referring to FIG. 12 and FIG. 13, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 11. Alternatively, the intervertebral prosthetic disc can be an intervertebral prosthetic disc according to any of the embodiments disclosed herein.

As shown in FIG. 12 and FIG. 13, the intervertebral prosthetic disc 400 is installed within the intervertebral space 214 that can be established between the superior vertebra 200 and the inferior vertebra 202 by removing vertebral disc material (not shown). In a particular embodiment, the superior keel 548 of the superior component 500 can at least partially engage the cancellous bone and cortical rim of the superior vertebra 200. Also, in a particular embodiment, the inferior keel 648 of the inferior component 600 can at least partially engage the cancellous bone and cortical rim of the inferior vertebra 202.

FIG. 13 indicates that the superior compliant structure 520 can engage the superior vertebra 200, e.g., the cortical rim and cancellous bone of the superior vertebra 200. The superior compliant structure 520 can mold, or otherwise form, to match the shape of the cortical rim and cancellous bone of the superior vertebra 200. In a particular embodiment, the superior compliant structure 520 can increase the contact area between the superior vertebra 200 and the superior support plate 502. As such, the superior compliant structure 520 can substantially reduce the contact stress between the superior vertebra 200 and the superior support plate 502.

Also, the inferior compliant structure 620 can engage the inferior vertebra 202, e.g., the cortical rim and cancellous bone of the inferior vertebra 202. The inferior compliant structure 620 can mold, or otherwise form, to match the shape of the cortical rim and cancellous bone of the inferior vertebra 200. In a particular embodiment, the inferior compliant structure 620 can increase the contact area between the inferior vertebra 200 and the inferior support plate 602. As such, the inferior compliant structure 620 can substantially reduce the contact stress between the inferior vertebra 200 and the inferior support plate 602.

After weight is applied to the segment of the spin in which the intervertebral prosthetic disc 400 is installed, the compliant structures 520, 620 can conform to the shape of the endplates in contact with the compliant structures 520, 620. In order to minimize the potential of subsidence, the endplates are preserved as much as possible, e.g., only the hyaline cartilage layer is removed from the endplates. Under load, the material within the compliant structures 520, 620 can flow within the compliant structures 520, 620 to allow the compliant structures to conform to the shape of the endplates. As such, contact between the vertebrae and the intervertebral prosthetic disc 400 is substantially maximized. Also, contact stress at non-conforming areas can be substantially reduced.

If a particular vertebral endplate has a slightly concave shape, the material within the adjacent compliant structure 520, 620 can flow toward the periphery of the compliant structure 520, 620. Also, if a particular vertebral endplate has a greater concave shape, the material within the adjacent compliant structure 520, 620 can flow away from the periphery of the compliant structure 520, 620. If a particular vertebral end plate has an irregular shape, the material within the adjacent compliant structure 520, 620 can flow within the compliant structure to conform to the irregular shape.

As illustrated in FIG. 12 and FIG. 13, the projection 508 that extends from the superior component 500 of the intervertebral prosthetic disc 400 can at least partially engage the depression 608 that is formed within the inferior component 600 of the intervertebral prosthetic disc 400. It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

In a particular embodiment, the intervertebral prosthetic disc 400 can allow angular movement in any radial direction relative to the intervertebral prosthetic disc 400. Further, as depicted in FIG. 13, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

Description of a Second Embodiment of an Intervertebral Prosthetic Disc

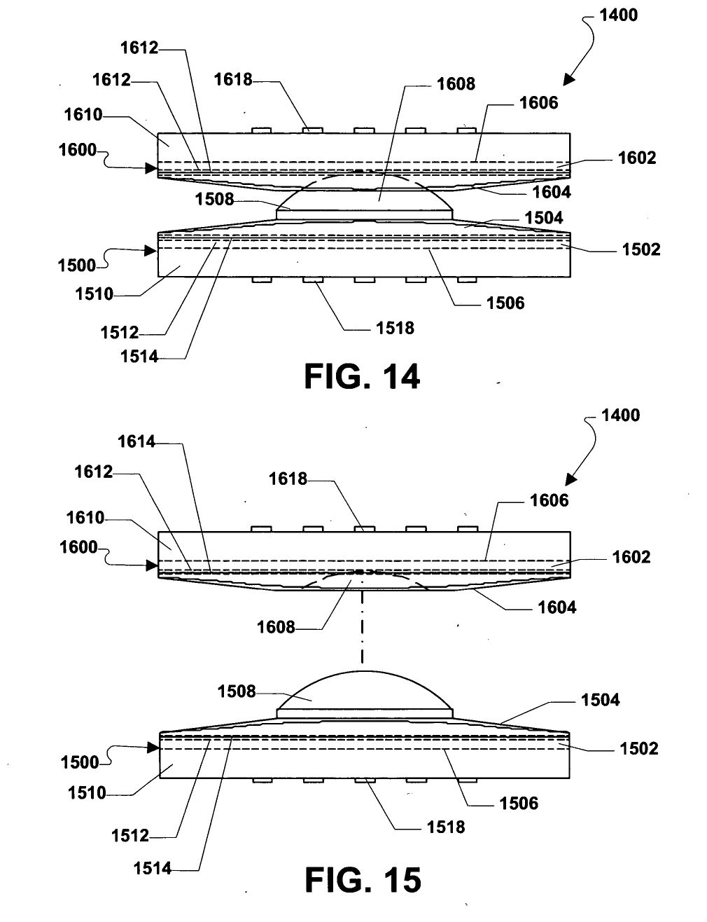

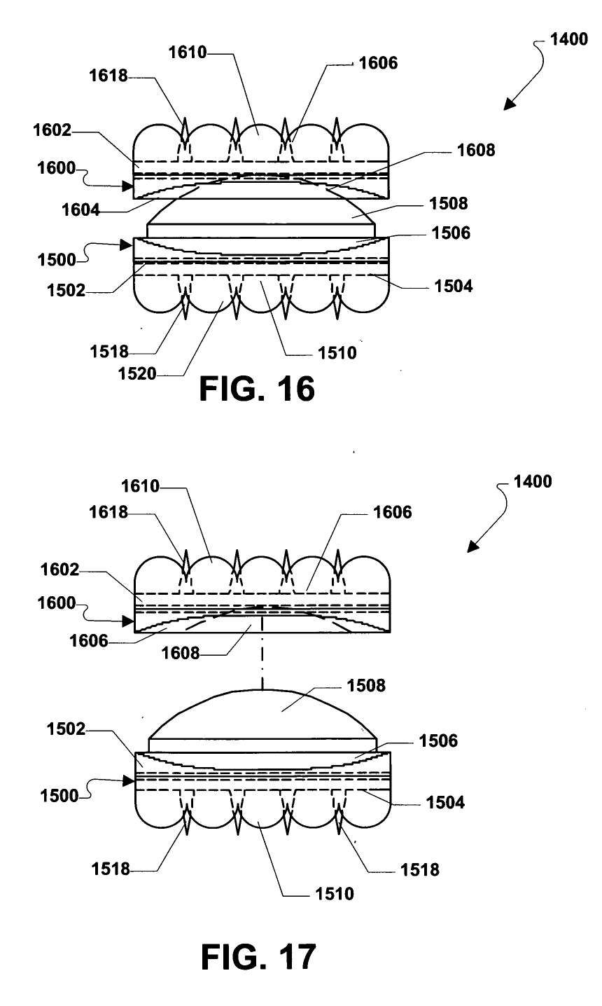

Referring to FIGS. 14 through 21 a second embodiment of an intervertebral prosthetic disc is shown and is generally designated 1400. As illustrated, the intervertebral prosthetic disc 1400 can include an inferior component 1500 and a superior component 1600. In a particular embodiment, the components 1500, 1600 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 1500, 1600 can be made from any other substantially rigid biocompatible materials.

In a particular embodiment, the inferior component 1500 can include an inferior support plate 1502 that has an inferior articular surface 1504 and an inferior bearing surface 1506. In a particular embodiment, the inferior articular surface 1504 can be generally rounded and the inferior bearing surface 1506 can be generally flat.

As illustrated in FIG. 14 through FIG. 21, a projection 1508 extends from the inferior articular surface 1504 of the inferior support plate 1502. In a particular embodiment, the projection 1508 has a hemi-spherical shape. Alternatively, the projection 1508 can have an elliptical shape, a cylindrical shape, or other arcuate shape.

As further illustrated, the inferior component 1500 can include an inferior compliant structure 1510 that can be affixed, or otherwise attached to the inferior component 1500. In a particular embodiment, a groove 1512 can be formed in the inferior component 1500, e.g., around the perimeter of the inferior component 1500. A wire 1514 can secure the inferior compliant structure 1510 within the groove 1512. For example, the ends of the wire 1514 may be laser welded to each other to create a permanent tension band.

In an alternative embodiment, the inferior compliant structure 1510 can be chemically bonded to the inferior bearing surface 1506, e.g., using an adhesive or another chemical bonding agent. Further, the inferior compliant structure 1510 can be mechanically anchored to the inferior bearing surface 1506, e.g., using hook-and-loop fasteners, or another type of fastener.

In a particular embodiment, after installation, the inferior compliant structure 1510 can be in direct contact with vertebral bone, e.g., cortical bone and cancellous bone. Further, in a particular embodiment, the inferior compliant structure 1510 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the inferior compliant structure 1510 can be made from a three-dimensional (3-D) woven structure, e.g., a three-dimensional (3-D) polyester structure. Further, in a particular embodiment, the inferior compliant structure 1510 can be resorbable, non-resorbable, or a combination thereof.

In a particular embodiment, the inferior compliant structure 1510 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant structure 1510 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 14 through FIG. 17 and FIG. 19 also show that the inferior component 1500 can include a plurality of inferior teeth 1518 that extend from the inferior bearing surface 1506. As shown, in a particular embodiment, the inferior teeth 1518 are generally saw-tooth, or triangle, shaped. Further, the inferior teeth 1518 are designed to engage cancellous bone of an inferior vertebra. Additionally, the inferior teeth 1518 can prevent the inferior component 1500 from moving with respect to an inferior vertebra after the intervertebral prosthetic disc 1400 is installed within the intervertebral space between the inferior vertebra and the superior vertebra.

In a particular embodiment, the inferior teeth 1518 can include other projections such as spikes, pins, blades, or a combination thereof that have any cross-sectional geometry.

In a particular embodiment, the inferior compliant structure 1510 can be reinforced where each inferior tooth 1518 protrudes therethrough. Further, the inferior teeth 1518 may not protrude through the inferior compliant structure 1510 until a load is placed on the intervertebral prosthetic disc 1400 and the inferior compliant structure 1510 conforms to the shape of the vertebra which the inferior compliant structure 1510 engages.

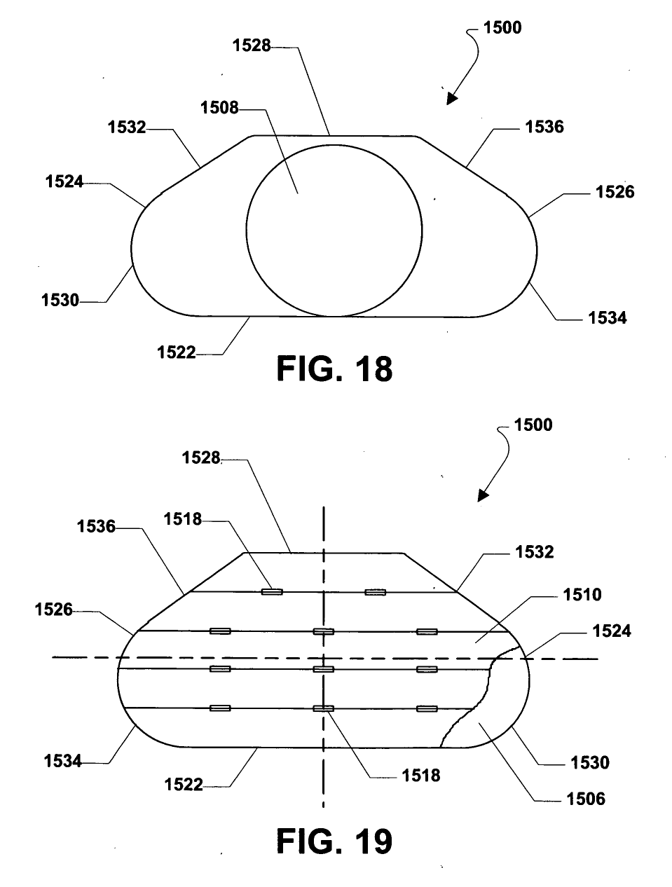

As illustrated in FIG. 18 and FIG. 19, the inferior component 1500 can be generally shaped to match the general shape of the vertebral body of a vertebra. For example, the inferior component 1500 can have a general trapezoid shape and the inferior component 1500 can include a posterior side 1522. A first lateral side 1524 and a second lateral side 1526 can extend from the posterior side 1522 to an anterior side 1528. In a particular embodiment, the first lateral side 1524 can include a curved portion 1530 and a straight portion 1532 that extends at an angle toward the anterior side 1528. Further, the second lateral side 1526 can also include a curved portion 1534 and a straight portion 1536 that extends at an angle toward the anterior side 1528.

As shown in FIG. 18 and FIG. 19, the anterior side 1528 of the inferior component 1500 can be relatively shorter than the posterior side 1522 of the inferior component 1500. Further, in a particular embodiment, the anterior side 1528 is substantially parallel to the posterior side 1522. As indicated in FIG. 18, the projection 1508 can be situated, or otherwise formed, on the inferior articular surface 1504 such that the perimeter of the projection 1508 is tangential to the posterior side 1522 of the inferior component 1500. In alternative embodiments (not shown), the projection 1508 can be situated, or otherwise formed, on the inferior articular surface 1504 such that the perimeter of the projection 1508 is tangential to the anterior side 1528 of the inferior component 1500 or tangential to both the anterior side 1528 and the posterior side 1522. In a particular embodiment, the projection 1508 and the inferior support plate 1502 comprise a monolithic body.

In a particular embodiment, the superior component 1600 can include a superior support plate 1602 that has a superior articular surface 1604 and a superior bearing surface 1606. In a particular embodiment, the superior articular surface 1604 can be generally rounded and the superior bearing surface 1606 can be generally flat.

As illustrated in FIG. 14 through FIG. 17 and FIG. 20, a depression 1608 extends into the superior articular surface 1604 of the superior support plate 1602. In a particular embodiment, the depression 1608 is sized and shaped to receive the projection 1508 of the inferior component 1500. For example, the depression 1608 can have a hemi-spherical shape. Alternatively, the depression 1608 can have an elliptical shape, a cylindrical shape, or other arcuate shape.

As further illustrated, the superior component 1600 can include a superior compliant structure 1610 that can be affixed, or otherwise attached to the superior component 1600. In a particular embodiment, a groove 1612 can be formed in the superior component 1600, e.g., around the perimeter of the superior component 1600. A wire 1614 can secure the superior compliant structure 1610 within the groove 1612. For example, the ends of the wire 1614 may be laser welded to each other to create a permanent tension band.

In an alternative embodiment, the superior compliant structure 1610 can be chemically bonded to the superior bearing surface 1606, e.g., using an adhesive or another chemical bonding agent. Further, the superior compliant structure 1610 can be mechanically anchored to the superior bearing surface 1606, e.g., using hook-and-loop fasteners, or another type of fastener.

In a particular embodiment, after installation, the superior compliant structure 1610 can be in direct contact with vertebral bone, e.g., cortical bone and cancellous bone. Further, in a particular embodiment, the superior compliant structure 1610 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the superior compliant structure 1610 can be made from a three-dimensional (3-D) woven structure, e.g., a three-dimensional (3-D) polyester structure. Further, in a particular embodiment, the superior compliant structure 1610 can be resorbable, non-resorbable, or a combination thereof.

In a particular embodiment, the superior compliant structure 1610 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant structure 1610 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 14 through FIG. 17 and FIG. 21 also show that the superior component 1600 can include a plurality of superior teeth 1618 that extend from the superior bearing surface 1606. As shown, in a particular embodiment, the superior teeth 1618 are generally saw-tooth, or triangle, shaped. Further, the superior teeth 1618 are designed to engage cancellous bone of a superior vertebra. Additionally, the superior teeth 1618 can prevent the superior component 1600 from moving with respect to a superior vertebra after the intervertebral prosthetic disc 1400 is installed within the intervertebral space between the superior vertebra and the superior vertebra.

In a particular embodiment, the superior teeth 1618 can include other projections such as spikes, pins, blades, or a combination thereof that have any cross-sectional geometry.

In a particular embodiment, the superior compliant structure 1610 can be reinforced where each superior tooth 1618 protrudes therethrough. Further, the superior teeth 1618 may not protrude through the superior compliant structure 1610 until a load is placed on the intervertebral prosthetic disc 1400 and the superior compliant structure 1610 conforms to the shape of the vertebra which the superior compliant structure 1610 engages.

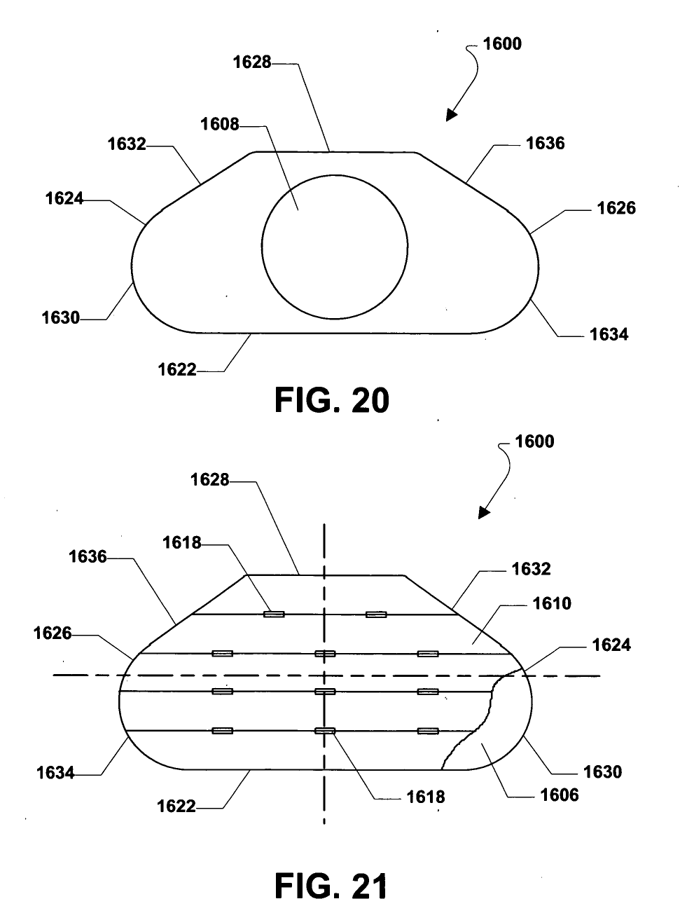

In a particular embodiment, the superior component 1600 can be shaped to match the shape of the inferior component 1500, shown in FIG. 18 and FIG. 19. Further, the superior component 1600 can be shaped to match the general shape of a vertebral body of a vertebra. For example, as shown in FIG. 20 and FIG. 21, the superior component 1600 can have a general trapezoid shape and the superior component 1600 can include a posterior side 1622. A first lateral side 1624 and a second lateral side 1626 can extend from the posterior side 1622 to an anterior side 1628. In a particular embodiment, the first lateral side 1624 can include a curved portion 1630 and a straight portion 1632 that extends at an angle toward the anterior side 1628. Further, the second lateral side 1626 can also include a curved portion 1634 and a straight portion 1636 that extends at an angle toward the anterior side 1628.

As shown in FIG. 20 and FIG. 21, the anterior side 1628 of the superior component 1600 can be relatively shorter than the posterior side 1622 of the superior component 1600. Further, in a particular embodiment, the anterior side 1628 is substantially parallel to the posterior side 1622.

In a particular embodiment, the overall height of the intervertebral prosthetic device 1400 can be in a range from six millimeters to twenty-two millimeters (6-22 mm). Further, the installed height of the intervertebral prosthetic device 1400 can be in a range from four millimeters to sixteen millimeters (4-16 mm). In a particular embodiment, the installed height can be substantially equivalent to the distance between an inferior vertebra and a superior vertebra when the intervertebral prosthetic device 1400 is installed there between.

In a particular embodiment, the length of the intervertebral prosthetic device 1400, e.g., along a longitudinal axis, can be in a range from thirty-three millimeters to fifty millimeters (33-50 mm). Additionally, the width of the intervertebral prosthetic device 1400, e.g., along a lateral axis, can be in a range from eighteen millimeters to twenty-nine millimeters (18-29 mm).

In a particular embodiment, the intervertebral prosthetic disc 1400 can be considered to be “low profile.” The low profile the intervertebral prosthetic device 1400 can allow the intervertebral prosthetic device 1400 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 1518, 1618 can be oriented to engage in a direction substantially opposite the direction of insertion of the prosthetic disc into the intervertebral space.

Further, the intervertebral prosthetic disc 1400 can have a general “bullet” shape as shown in the posterior plan view, described herein. The bullet shape of the intervertebral prosthetic disc 1400 can further allow the intervertebral prosthetic disc 1400 to be inserted through the patient's psoas muscle while minimizing risk to the patient's spinal cord and sympathetic chain.

Description of a Third Embodiment of an Intervertebral Prosthetic Disc

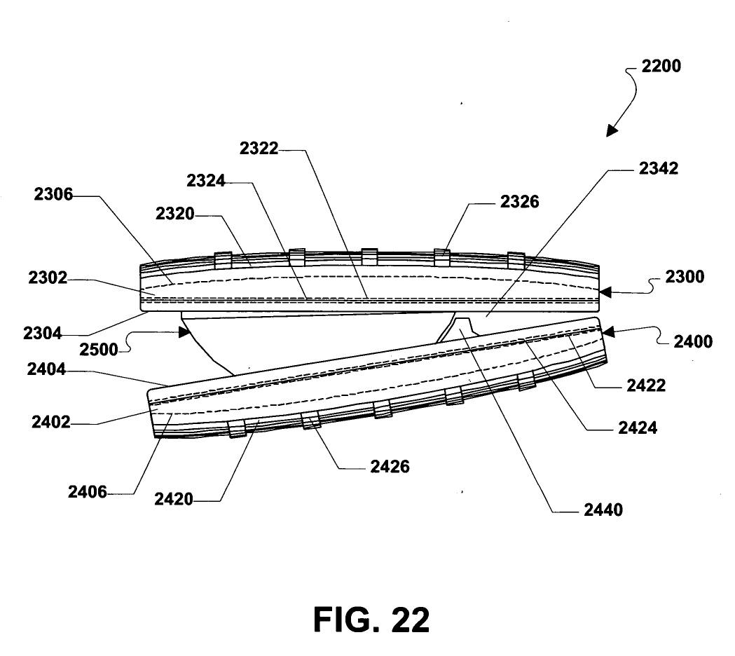

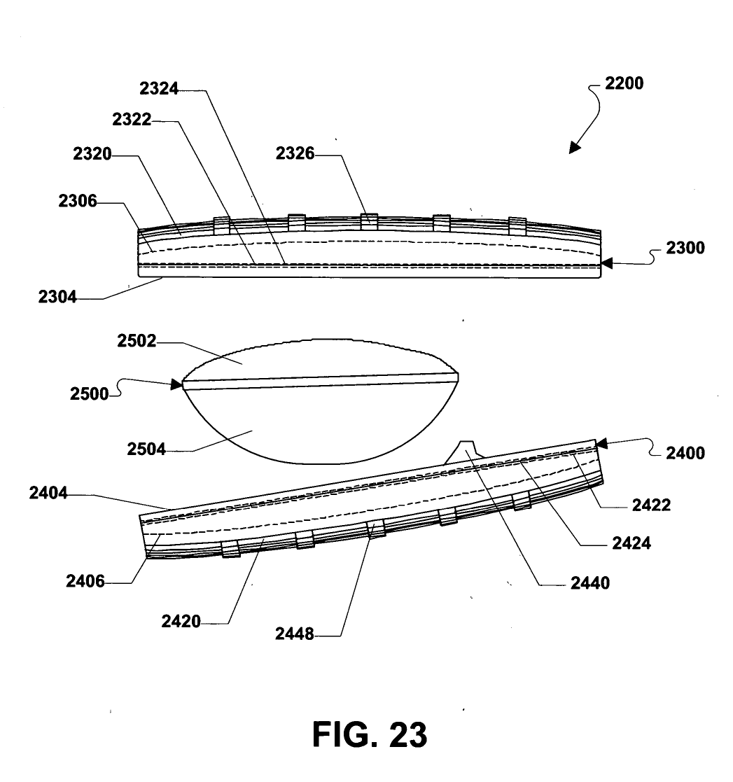

Referring to FIGS. 22 through 26 a third embodiment of an intervertebral prosthetic disc is shown and is generally designated 2200. As illustrated, the intervertebral prosthetic disc 2200 can include a superior component 2300, an inferior component 2400, and a nucleus 2500 disposed, or otherwise installed, there between. In a particular embodiment, the components 2300, 2400 and the nucleus 2500 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 2300, 2400 can be made from any other substantially rigid biocompatible materials.

In a particular embodiment, the superior component 2300 can include a superior support plate 2302 that has a superior articular surface 2304 and a superior bearing surface 2306. In a particular embodiment, the superior articular surface 2304 can be substantially flat and the superior bearing surface 2306 can be generally curved. In an alternative embodiment, at least a portion of the superior articular surface 2304 can be generally curved and the superior bearing surface 2306 can be substantially flat.

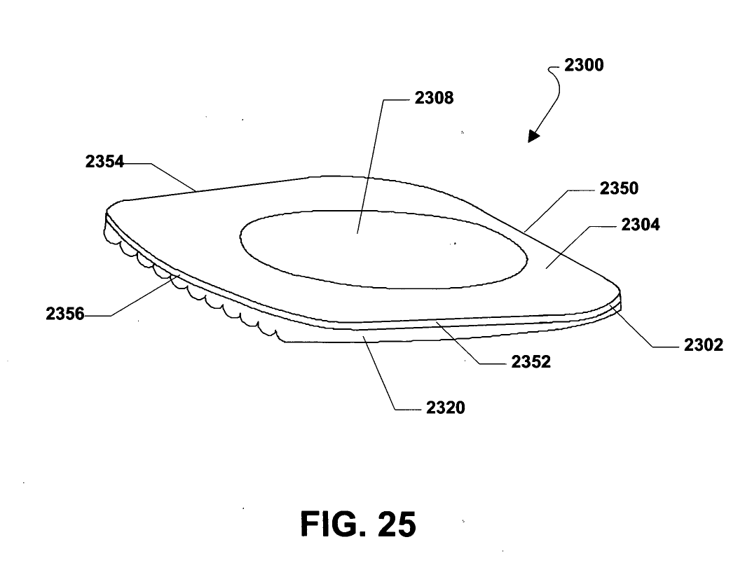

As illustrated in FIG. 25, a superior depression 2308 is established within the superior articular surface 2304 of the superior support plate 2302. In a particular embodiment, the superior depression 2308 has an arcuate shape. For example, the superior depression 2308 can have a hemispherical shape, an elliptical shape, a cylindrical shape, or any combination thereof.

As further illustrated, the superior component 2300 can include a superior compliant structure 2320 that can be affixed, or otherwise attached to the superior component 2300. In a particular embodiment, a groove 2322 can be formed in the superior component 2300, e.g., around the perimeter of the superior component 2300. A wire 2324 can secure the superior compliant structure 2320 within the groove 2322. For example, the ends of the wire 2324 may be laser welded to each other to create a permanent tension band.

In an alternative embodiment, the superior compliant structure 2320 can be chemically bonded to the superior bearing surface 2306, e.g., using an adhesive or another chemical bonding agent. Further, the superior compliant structure 2320 can be mechanically anchored to the superior bearing surface 2306, e.g., using hook-and-loop fasteners, or another type of fastener.

In a particular embodiment, after installation, the superior compliant structure 2320 can be in direct contact with vertebral bone, e.g., cortical bone and cancellous bone. Further, in a particular embodiment, the superior compliant structure 2320 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the superior compliant structure 2320 can be made from a three-dimensional (3-D) woven structure, e.g., a three-dimensional (3-D) polyester structure. Further, in a particular embodiment, the superior compliant structure 2320 can be resorbable, non-resorbable, or a combination thereof.

In a particular embodiment, the superior compliant structure 2320 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof

In a particular embodiment, the superior compliant structure 2320 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 22 through FIG. 24 show that the superior component 2300 can also include a plurality of superior teeth 2326 that extend from the superior bearing surface 2306. As shown, in a particular embodiment, the superior teeth 2326 are generally saw-tooth, or triangle, shaped. Further, the superior teeth 2326 are designed to engage cancellous bone of a superior vertebra. Additionally, the superior teeth 2318 can prevent the superior component 2300 from moving with respect to a superior vertebra after the intervertebral prosthetic disc 2300 is installed within the intervertebral space between the superior vertebra and the superior vertebra.

In a particular embodiment, the superior teeth 2326 can include other projections such as spikes, pins, blades, or a combination thereof that have any cross-sectional geometry.

In a particular embodiment, the superior compliant structure 2320 can be reinforced where each superior tooth 2326 protrudes therethrough. Further, the superior teeth 2326 may not protrude through the superior compliant structure 2320 until a load is placed on the intervertebral prosthetic disc 1400 and the superior compliant structure 2320 conforms to the shape of the vertebra which the superior compliant structure 2320 engages.

In a particular embodiment, the superior component 2300, depicted in FIG. 25, can be generally rectangular in shape. For example, the superior component 2300 can have a substantially straight posterior side 2350. A first substantially straight lateral side 2352 and a second substantially straight lateral side 2354 can extend substantially perpendicularly from the posterior side 2350 to an anterior side 2356. In a particular embodiment, the anterior side 2356 can curve outward such that the superior component 2300 is wider through the middle than along the lateral sides 2352, 2354. Further, in a particular embodiment, the lateral sides 2352, 2354 are substantially the same length.

In a particular embodiment, the inferior component 2400 can include an inferior support plate 2402 that has an inferior articular surface 2404 and an inferior bearing surface 2406. In a particular embodiment, the inferior articular surface 2404 can be substantially flat and the inferior bearing surface 2406 can be generally curved. In an alternative embodiment, at least a portion of the inferior articular surface 2404 can be generally curved and the inferior bearing surface 2406 can be substantially flat.

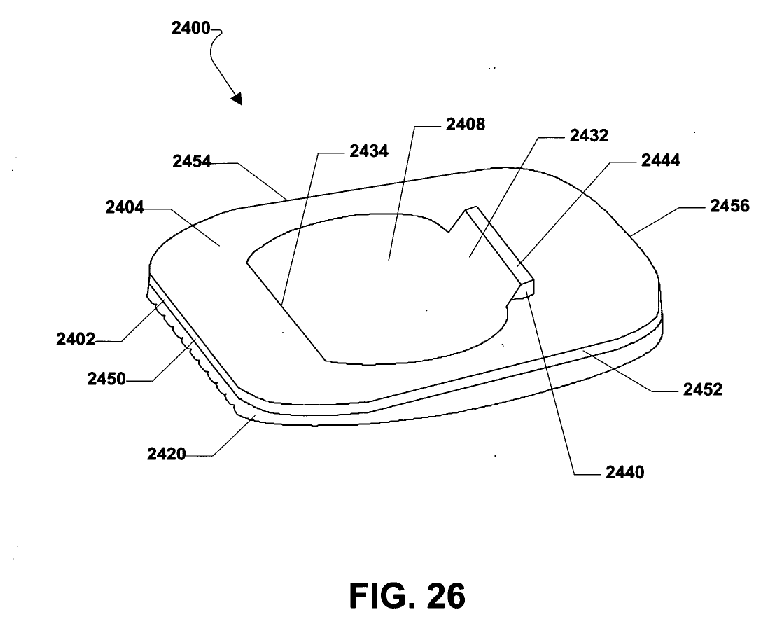

As illustrated in FIG. 26, an inferior depression 2408 is established within the inferior articular surface 2404 of the inferior support plate 2402. In a particular embodiment, the inferior depression 2408 has an arcuate shape. For example, the inferior depression 2408 can have a hemispherical shape, an elliptical shape, a cylindrical shape, or any combination thereof.

As further illustrated, the inferior component 2400 can include an inferior compliant structure 2420 that can be affixed, or otherwise attached to the inferior component 2400. In a particular embodiment, a groove 2422 can be formed in the inferior component 2400, e.g., around the perimeter of the inferior component 2400. A wire 2424 can secure the inferior compliant structure 2420 within the groove 2422. For example, the ends of the wire 2424 may be laser welded to each other to create a permanent tension band.

In an alternative embodiment, the inferior compliant structure 2420 can be chemically bonded to the inferior bearing surface 2406, e.g., using an adhesive or another chemical bonding agent. Further, the inferior compliant structure 2420 can be mechanically anchored to the inferior bearing surface 2406, e.g., using hook-and-loop fasteners, or another type of fastener.

In a particular embodiment, after installation, the inferior compliant structure 2420 can be in direct contact with vertebral bone, e.g., cortical bone and cancellous bone. Further, in a particular embodiment, the inferior compliant structure 2420 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the inferior compliant structure 2420 can be made from a three-dimensional (3-D) woven structure, e.g., a three-dimensional (3-D) polyester structure. Further, in a particular embodiment, the inferior compliant structure 2420 can be resorbable, non-resorbable, or a combination thereof.

In a particular embodiment, the inferior compliant structure 2420 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant structure 2420 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 22 through FIG. 24 show that the inferior component 2400 can also include a plurality of inferior teeth 2426 that extend from the inferior bearing surface 2406. As shown, in a particular embodiment, the inferior teeth 2426 are generally saw-tooth, or triangle, shaped. Further, the inferior teeth 2426 are designed to engage cancellous bone of an inferior vertebra. Additionally, the inferior teeth 2418 can prevent the inferior component 2400 from moving with respect to an inferior vertebra after the intervertebral prosthetic disc 2400 is installed within the intervertebral space between the inferior vertebra and the inferior vertebra.

In a particular embodiment, the inferior teeth 2426 can include other projections such as spikes, pins, blades, or a combination thereof that have any cross-sectional geometry.

In a particular embodiment, the inferior compliant structure 2420 can be reinforced where each inferior tooth 2426 protrudes therethrough. Further, the inferior teeth 2426 may not protrude through the inferior compliant structure 2420 until a load is placed on the intervertebral prosthetic disc 1400 and the inferior compliant structure 2420 conforms to the shape of the vertebra which the inferior compliant structure 2420 engages.

As further shown in FIG. 26, the inferior depression 2408 can include an anterior rim 2432 and a poster rim 2434. Further, an inferior nucleus containment rail 2440 extends from the inferior articular surface 2404 adjacent to the anterior rim 2432 of the inferior depression 2408. As shown in FIG. 26, the inferior nucleus containment rail 2440 is an extension of the surface of the inferior depression 2408. In a particular embodiment, as shown in FIG. 22, the inferior nucleus containment rail 2440 extends into a gap 2442 that can be established between the superior component 2300 and the inferior component 2400 posterior to the nucleus 2500. Further, the inferior nucleus containment rail 2440 can include a slanted upper surface 2444. In a particular embodiment, the slanted upper surface 2444 of the inferior nucleus containment rail 2440 can prevent the inferior nucleus containment rail 2440 from interfering with the motion of the superior component 2300 with respect to the inferior component 2400.

In lieu of, or in addition to, the inferior nucleus containment rail 2440, a superior nucleus containment rail (not shown) can extend from the superior articular surface 2304 of the superior component 2300. In a particular embodiment, the superior nucleus containment rail (not shown) can be configured substantially identical to the inferior nucleus containment rail 2440. In various alternative embodiments (not shown), each or both of the superior component 2300 and the inferior component 2400 can include multiple nucleus containment rails extending from the respective articular surfaces 2304, 2404. The containment rails can be staggered or provided in other configurations based on the perceived need to prevent nucleus migration in a given direction.

In a particular embodiment, the inferior component 2400, shown in FIG. 26, can be shaped to match the shape of the superior component 2300, shown in FIG. 25. Further, the inferior component 2400 can be generally rectangular in shape. For example, the inferior component 2400 can have a substantially straight posterior side 2450. A first substantially straight lateral side 2452 and a second substantially straight lateral side 2454 can extend substantially perpendicularly from the posterior side 2450 to an anterior side 2456. In a particular embodiment, the anterior side 2456 can curve outward such that the inferior component 2400 is wider through the middle than along the lateral sides 2452, 2454. Further, in a particular embodiment, the lateral sides 2452, 2454 are substantially the same length.

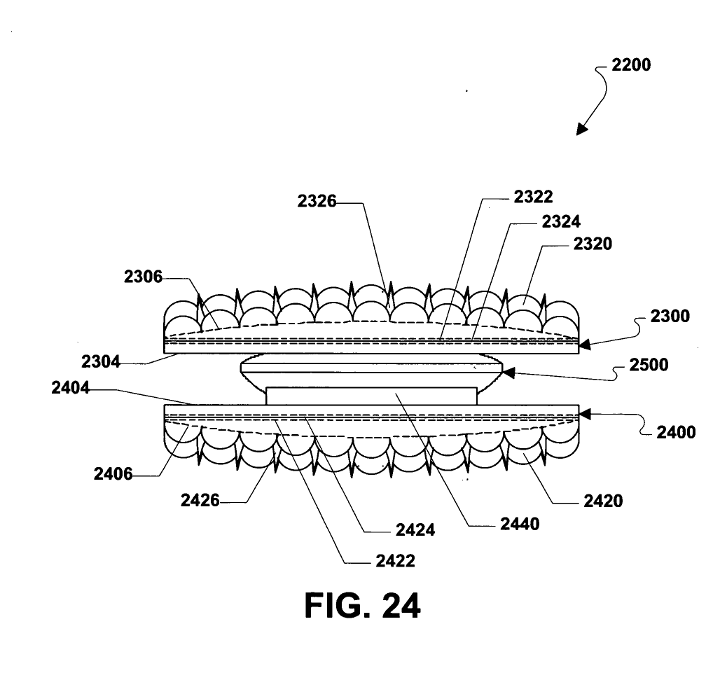

FIG. 24 shows that the nucleus 2500 can include a superior bearing surface 2502 and an inferior bearing surface 2504. In a particular embodiment, the superior bearing surface 2502 and the inferior bearing surface 2504 can each have an arcuate shape. For example, the superior bearing surface 2502 of the nucleus 2500 and the inferior bearing surface 2504 of the nucleus 2500 can have a hemispherical shape, an elliptical shape, a cylindrical shape, or any combination thereof. Further, in a particular embodiment, the superior bearing surface 2502 can be curved to match the superior depression 2308 of the superior component 2300. Also, in a particular embodiment, the inferior bearing surface 2504 of the nucleus can be curved to match the inferior depression 2408 of the inferior component 2400.

As shown in FIG. 22, the superior bearing surface 2502 of the nucleus 2500 can engage the superior depression 2308 and allow the superior component 2300 to move relative to the nucleus 2500. Also, the inferior bearing surface 2504 of the nucleus 2500 can engage the inferior depression 2408 and allow the inferior component 2400 to move relative to the nucleus 2500. Accordingly, the nucleus 2500 can engage the superior component 2300 and the inferior component 2400 and the nucleus 2500 can allow the superior component 2300 to rotate with respect to the inferior component 2400.

In a particular embodiment, the inferior nucleus containment rail 2430 on the inferior component 2400 can prevent the nucleus 2500 from migrating, or moving, with respect to the superior component 2300, the inferior component 2400, or a combination thereof. In other words, the inferior nucleus containment rail 2430 can prevent the nucleus 2500 from moving out of the superior depression 2308, the inferior depression 2408, or a combination thereof.

Further, the inferior nucleus containment rail 2430 can prevent the nucleus 2500 from being expelled from the intervertebral prosthetic device 2200. In other words, the inferior nucleus containment rail 2430 on the inferior component 2400 can prevent the nucleus 2500 from being completely ejected from the intervertebral prosthetic device 2200 while the superior component 2300 and the inferior component 2400 move with respect to each other.

In a particular embodiment, the overall height of the intervertebral prosthetic device 2200 can be in a range from fourteen millimeters to forty-six millimeters (14-46 mm). Further, the installed height of the intervertebral prosthetic device 2200 can be in a range from eight millimeters to sixteen millimeters (8-16 mm). In a particular embodiment, the installed height can be substantially equivalent to the distance between an inferior vertebra and a superior vertebra when the intervertebral prosthetic device 2200 is installed there between.

In a particular embodiment, the length of the intervertebral prosthetic device 2200, e.g., along a longitudinal axis, can be in a range from thirty millimeters to forty millimeters (30-40 mm). Additionally, the width of the intervertebral prosthetic device 2200, e.g., along a lateral axis, can be in a range from twenty-five millimeters to forty millimeters (25-40 mm).

Description of a Fourth Embodiment of an Intervertebral Prosthetic Disc

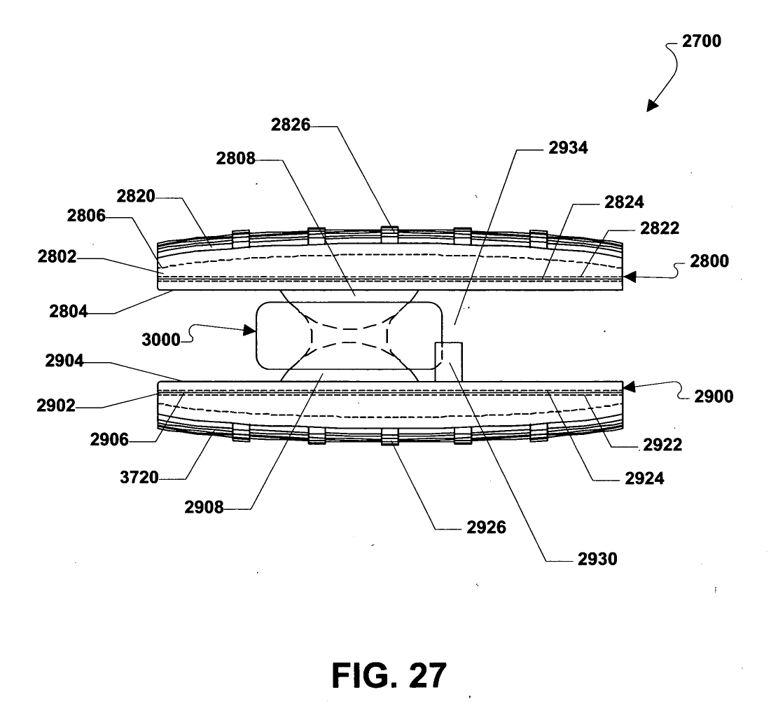

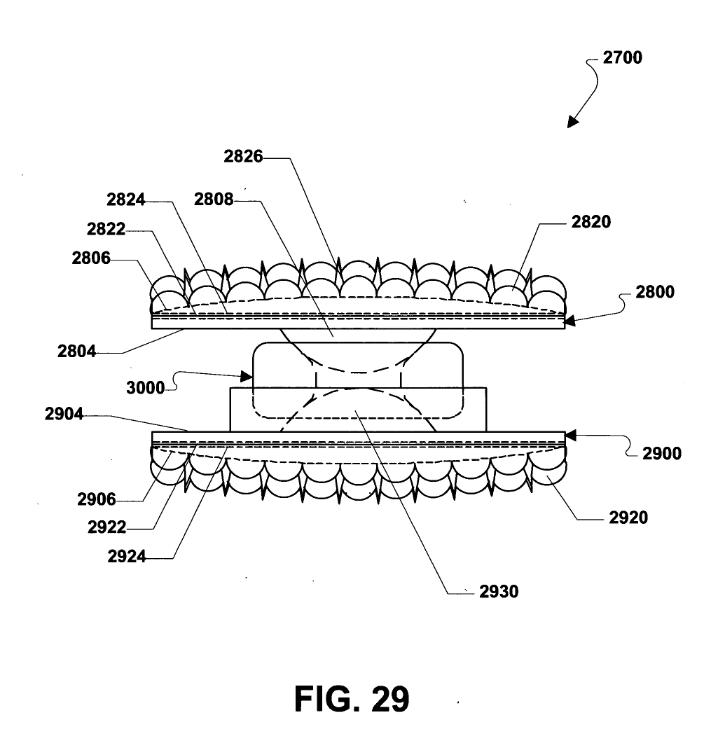

Referring to FIGS. 27 through 31, a fourth embodiment of an intervertebral prosthetic disc is shown and is generally designated 2700. As illustrated, the intervertebral prosthetic disc 2700 can include a superior component 2800, an inferior component 2900, and a nucleus 3000 disposed, or otherwise installed, there between. In a particular embodiment, the components 2800, 2900 and the nucleus 3000 can be made from one or more extended use biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 2800, 2900 can be made from any other substantially rigid biocompatible materials.

In a particular embodiment, the superior component 2800 can include a superior support plate 2802 that has a superior articular surface 2804 and a superior bearing surface 2806. In a particular embodiment, the superior articular surface 2804 can be substantially flat and the superior bearing surface 2806 can be generally curved. In an alternative embodiment, at least a portion of the superior articular surface 2804 can be generally curved and the superior bearing surface 2806 can be substantially flat.

As illustrated in FIG. 27 through FIG. 30, a superior projection 2808 extends from the superior articular surface 2804 of the superior support plate 2802. In a particular embodiment, the superior projection 2808 has an arcuate shape. For example, the superior depression 2808 can have a hemispherical shape, an elliptical shape, a cylindrical shape, or any combination thereof.

As further illustrated, the superior component 2800 can include a superior compliant structure 2820 that can be affixed, or otherwise attached to the superior component 2800. In a particular embodiment, a groove 2822 can be formed in the superior component 2800, e.g., around the perimeter of the superior component 2800. A wire 2824 can secure the superior compliant structure 2820 within the groove 2822. For example, the ends of the wire 2824 may be laser welded to each other to create a permanent tension band.

In an alternative embodiment, the superior compliant structure 2820 can be chemically bonded to the superior bearing surface 2806, e.g., using an adhesive or another chemical bonding agent. Further, the superior compliant structure 2820 can be mechanically anchored to the superior bearing surface 2806, e.g., using hook-and-loop fasteners, or another type of fastener.

In a particular embodiment, after installation, the superior compliant structure 2820 can be in direct contact with vertebral bone, e.g., cortical bone and cancellous bone. Further, in a particular embodiment, the superior compliant structure 2820 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the superior compliant structure 2820 can be made from a three-dimensional (3-D) woven structure, e.g., a three-dimensional (3-D) polyester structure. Further, in a particular embodiment, the superior compliant structure 2820 can be resorbable, non-resorbable, or a combination thereof.

In a particular embodiment, the superior compliant structure 2820 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the superior compliant structure 2820 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 22 through FIG. 24 show that the superior component 2800 can also include a plurality of superior teeth 2826 that extend from the superior bearing surface 2806. As shown, in a particular embodiment, the superior teeth 2826 are generally saw-tooth, or triangle, shaped. Further, the superior teeth 2826 are designed to engage cancellous bone of a superior vertebra. Additionally, the superior teeth 2818 can prevent the superior component 2800 from moving with respect to a superior vertebra after the intervertebral prosthetic disc 2800 is installed within the intervertebral space between the superior vertebra and the superior vertebra.

In a particular embodiment, the superior teeth 2826 can include other projections such as spikes, pins, blades, or a combination thereof that have any cross-sectional geometry.

In a particular embodiment, the superior compliant structure 2820 can be reinforced where each superior tooth 2826 protrudes therethrough. Further, the superior teeth 2826 may not protrude through the superior compliant structure 2820 until a load is placed on the intervertebral prosthetic disc 1400 and the superior compliant structure 2820 conforms to the shape of the vertebra which the superior compliant structure 2820 engages.

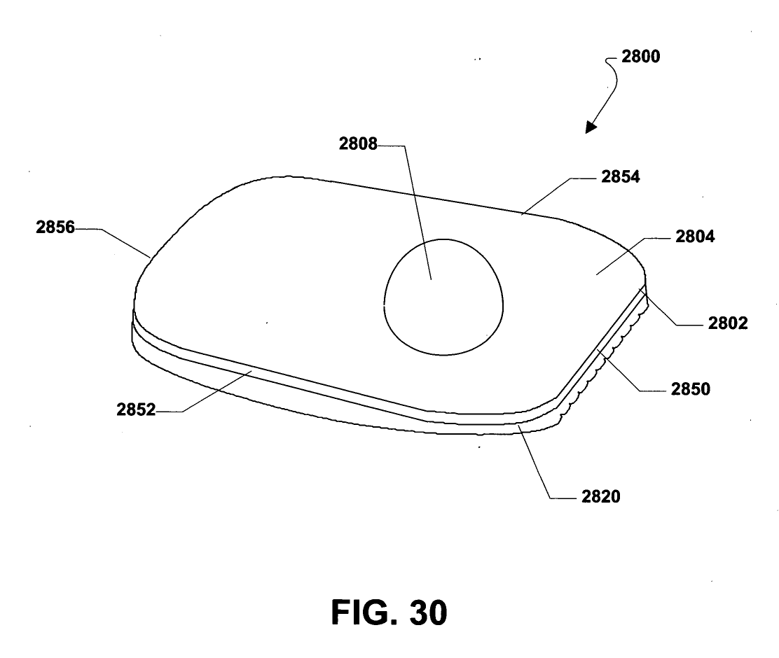

In a particular embodiment, the superior component 2800, depicted in FIG. 30, can be generally rectangular in shape. For example, the superior component 2800 can have a substantially straight posterior side 2850. A first substantially straight lateral side 2852 and a second substantially straight lateral side 2854 can extend substantially perpendicularly from the posterior side 2850 to an anterior side 2856. In a particular embodiment, the anterior side 2856 can curve outward such that the superior component 2800 is wider through the middle than along the lateral sides 2852, 2854. Further, in a particular embodiment, the lateral sides 2852, 2854 are substantially the same length.

In a particular embodiment, the inferior component 2900 can include an inferior support plate 2902 that has an inferior articular surface 2904 and an inferior bearing surface 2906. In a particular embodiment, the inferior articular surface 2904 can be substantially flat and the inferior bearing surface 2906 can be generally curved. In an alternative embodiment, at least a portion of the inferior articular surface 2904 can be generally curved and the inferior bearing surface 2906 can be substantially flat.

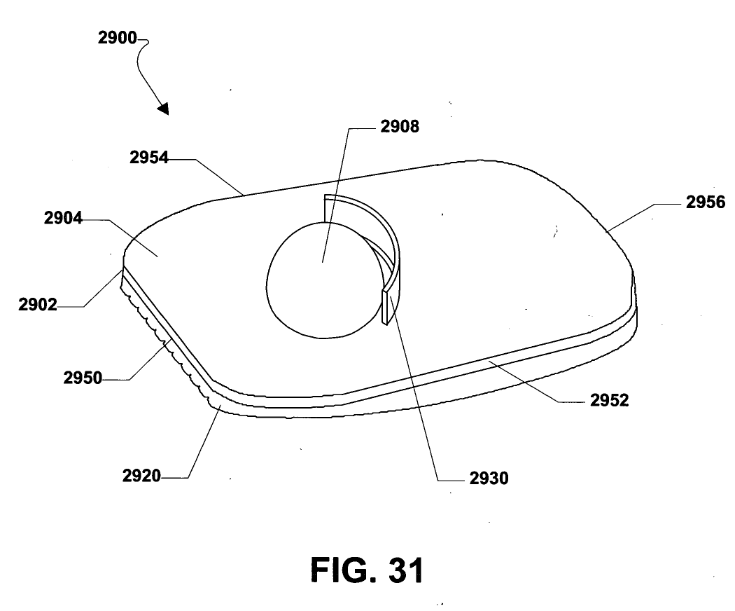

As illustrated in FIG. 31, an inferior projection 2908 can extend from the inferior articular surface 2904 of the inferior support plate 2902. In a particular embodiment, the inferior projection 2908 has an arcuate shape. For example, the inferior projection 2908 can have a hemispherical shape, an elliptical shape, a cylindrical shape, or any combination thereof.

As further illustrated, the inferior component 2400 can include an inferior compliant structure 2420 that can be affixed, or otherwise attached to the inferior component 2400. In a particular embodiment, a groove 2422 can be formed in the inferior component 2400, e.g., around the perimeter of the inferior component 2400. A wire 2424 can secure the inferior compliant structure 2420 within the groove 2422. For example, the ends of the wire 2424 may be laser welded to each other to create a permanent tension band.

In an alternative embodiment, the inferior compliant structure 2420 can be chemically bonded to the inferior bearing surface 2406, e.g., using an adhesive or another chemical bonding agent. Further, the inferior compliant structure 2420 can be mechanically anchored to the inferior bearing surface 2406, e.g., using hook-and-loop fasteners, or another type of fastener.

In a particular embodiment, after installation, the inferior compliant structure 2420 can be in direct contact with vertebral bone, e.g., cortical bone and cancellous bone. Further, in a particular embodiment, the inferior compliant structure 2420 can be a fabric structure having a plurality of adjacent, generally cylindrical tubes. The tubes of the fabric structure may be interconnected to allow fluid to flow there between. In a particular embodiment, the fabric structure can made from be poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof. Alternatively, the inferior compliant structure 2420 can be made from a three-dimensional (3-D) woven structure, e.g., a three-dimensional (3-D) polyester structure. Further, in a particular embodiment, the inferior compliant structure 2420 can be resorbable, non-resorbable, or a combination thereof.

In a particular embodiment, the inferior compliant structure 2420 can be filled with an extended use biocompatible material. For example, the extended use biocompatible materials can include synthetic polymers, natural polymers, bioactive ceramics, carbon nanofibers, or combinations thereof.

In a particular embodiment, the synthetic polymers can include polyurethane materials, polyolefin materials, polyether materials, polyester materials, polycarbonate materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. The polyester materials can include polylactide. The polycarbonate materials can include tyrosine polycarbonate.

In a particular embodiment, the natural polymers can include collagen, gelatin, fibrin, keratin, chitosan, chitin, hyaluronic acid, albumin, silk, elastin, or a combination thereof. Further, in a particular embodiment, the bioactive ceramics can include hydroxyapatite (HA), hydroxyapatite tricalcium phosphate (HATCP), calcium phosphate, calcium sulfate, or a combination thereof.

In a particular embodiment, the inferior compliant structure 2420 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

FIG. 22 through FIG. 24 show that the inferior component 2400 can also include a plurality of inferior teeth 2426 that extend from the inferior bearing surface 2406. As shown, in a particular embodiment, the inferior teeth 2426 are generally saw-tooth, or triangle, shaped. Further, the inferior teeth 2426 are designed to engage cancellous bone of an inferior vertebra. Additionally, the inferior teeth 2418 can prevent the inferior component 2400 from moving with respect to an inferior vertebra after the intervertebral prosthetic disc 2400 is installed within the intervertebral space between the inferior vertebra and the inferior vertebra.

In a particular embodiment, the inferior teeth 2426 can include other projections such as spikes, pins, blades, or a combination thereof that have any cross-sectional geometry.

In a particular embodiment, the inferior compliant structure 2420 can be reinforced where each inferior tooth 2426 protrudes therethrough. Further, the inferior teeth 2426 may not protrude through the inferior compliant structure 2420 until a load is placed on the intervertebral prosthetic disc 1400 and the inferior compliant structure 2420 conforms to the shape of the vertebra which the inferior compliant structure 2420 engages.

As further shown, an inferior nucleus containment rail 2930 can extend from the inferior articular surface 2904 adjacent to the inferior projection 2908. As shown in FIG. 31, the inferior nucleus containment rail 2930 is a curved wall that extends from the inferior articular surface 2904. In a particular embodiment, the inferior nucleus containment rail 2930 can be curved to match the shape, or curvature, of the inferior projection 2908. Alternatively, the inferior nucleus containment rail 2930 can be curved to match the shape, or curvature, of the nucleus 3000. In a particular embodiment, the inferior nucleus containment rail 2930 extends into a gap 2934 that can be established between the superior component 2800 and the inferior component 2900 posterior to the nucleus 3000.

In lieu of, or in addition to, the inferior nucleus containment rail 2930, a superior nucleus containment rail (not shown) can extend from the superior articular surface 2804 of the superior component 2800. In a particular embodiment, the superior nucleus containment rail (not shown) can be configured substantially identical to the inferior nucleus containment rail 2930. In various alternative embodiments (not shown), each or both of the superior component 2800 and the inferior component 2900 can include multiple nucleus containment rails extending from the respective articular surfaces 2804, 2904. The containment rails can be staggered or provided in other configurations based on the perceived need to prevent nucleus migration in a given direction.

In a particular embodiment, the inferior component 2900, shown in FIG. 31, can be shaped to match the shape of the superior component 2800, shown in FIG. 30. Further, the inferior component 2900 can be generally rectangular in shape. For example, the inferior component 2900 can have a substantially straight posterior side 2950. A first substantially straight lateral side 2952 and a second substantially straight lateral side 2954 can extend substantially perpendicularly from the posterior side 2950 to an anterior side 2956. In a particular embodiment, the anterior side 2956 can curve outward such that the inferior component 2900 is wider through the middle than along the lateral sides 2952, 2954. Further, in a particular embodiment, the lateral sides 2952, 2954 are substantially the same length.

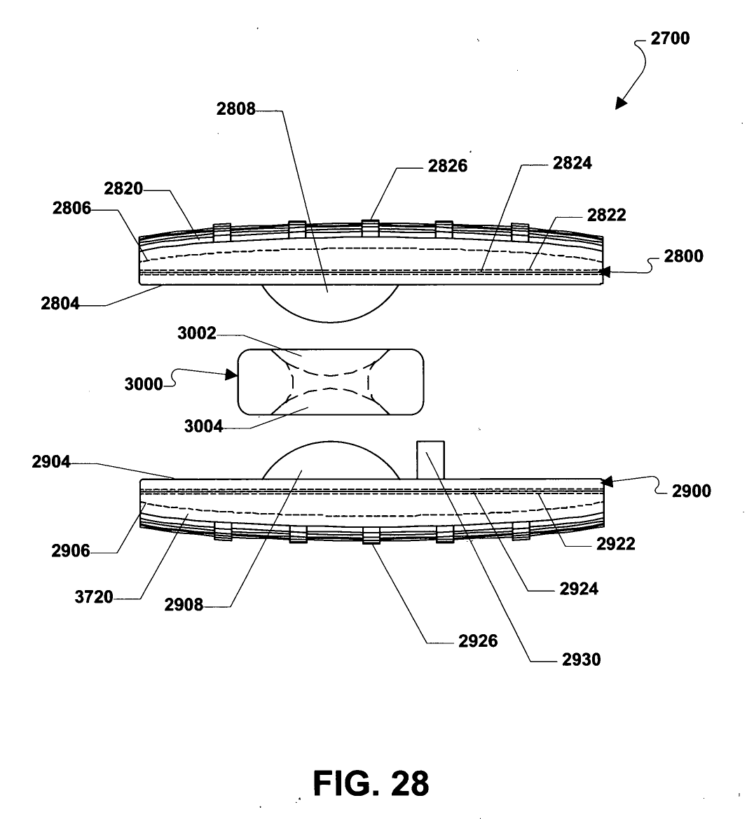

FIG. 28 shows that the nucleus 3000 can include a superior depression 3002 and an inferior depression 3004. In a particular embodiment, the superior depression 3002 and the inferior depression 3004 can each have an arcuate shape. For example, the superior depression 3002 of the nucleus 3000 and the inferior depression 3004 of the nucleus 3000 can have a hemispherical shape, an elliptical shape, a cylindrical shape, or any combination thereof. Further, in a particular embodiment, the superior depression 3002 can be curved to match the superior projection 2808 of the superior component 2800. Also, in a particular embodiment, the inferior depression 3004 of the nucleus 3000 can be curved to match the inferior projection 2908 of the inferior component 2900.

As shown in FIG. 27, the superior depression 3002 of the nucleus 3000 can engage the superior projection 2808 and allow the superior component 2800 to move relative to the nucleus 3000. Also, the inferior depression 3004 of the nucleus 3000 can engage the inferior projection 2908 and allow the inferior component 2900 to move relative to the nucleus 3000. Accordingly, the nucleus 3000 can engage the superior component 2800 and the inferior component 2900, and the nucleus 3000 can allow the superior component 2800 to rotate with respect to the inferior component 2900.

In a particular embodiment, the inferior nucleus containment rail 2930 on the inferior component 2900 can prevent the nucleus 3000 from migrating, or moving, with respect to the superior component 2800 and the inferior component 2900. In other words, the inferior nucleus containment rail 2930 can prevent the nucleus 3000 from moving off of the superior projection 2808, the inferior projection 2908, or a combination thereof.

Further, the inferior nucleus containment rail 2930 can prevent the nucleus 3000 from being expelled from the intervertebral prosthetic device 2700. In other words, the inferior nucleus containment rail 2930 on the inferior component 2900 can prevent the nucleus 3000 from being completely ejected from the intervertebral prosthetic device 2700 while the superior component 2800 and the inferior component 2900 move with respect to each other.

In a particular embodiment, the overall height of the intervertebral prosthetic device 2700 can be in a range from fourteen millimeters to forty-six millimeters (14-46 mm). Further, the installed height of the intervertebral prosthetic device 2700 can be in a range from eight millimeters to sixteen millimeters (8-16 mm). In a particular embodiment, the installed height can be substantially equivalent to the distance between an inferior vertebra and a superior vertebra when the intervertebral prosthetic device 2700 is installed there between.

In a particular embodiment, the length of the intervertebral prosthetic device 2700, e.g., along a longitudinal axis, can be in a range from thirty millimeters to forty millimeters (30-40 mm). Additionally, the width of the intervertebral prosthetic device 2700, e.g., along a lateral axis, can be in a range from twenty-five millimeters to forty millimeters (25-40 mm).

### Conclusion

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

The compliant structures of the intervertebral prosthetic disc can allow the intervertebral prosthetic disc to conform to the shapes of the vertebrae between which the intervertebral prosthetic disc is implanted. Full conformance can increase the surface area for osteointegration, which, in turn, can prevent, or substantially minimize, the chance of the intervertebral prosthetic disc becoming loose during the lifetime of the intervertebral prosthetic disc.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc to be installed within an intervertebral space between a first vertebra and a second vertebra, the intervertebral prosthetic disc comprising:
a first component having a first compliant structure configured to engage the first vertebra and at least partially conform to a shape of the first vertebra; and
a second component configured to engage the second vertebra.

2. The intervertebral prosthetic disc of claim 1, wherein the first compliant structure comprises a first fabric structure filled with an extended use biocompatible material.

3. The intervertebral prosthetic disc of claim 2, wherein the first fabric structure includes a plurality of adjacent cylindrical tubes.

4. The intervertebral prosthetic disc of claim 3, wherein the plurality of adjacent cylindrical tubes are interconnected to allow the extended use biocompatible material to flow there between.

5. The intervertebral prosthetic disc of claim 4, wherein the second component includes a second compliant structure configured to engage the second vertebra and as least partially conform to a shape of the second vertebra.

6. The intervertebral prosthetic disc of claim 5, wherein the second compliant structure comprises a second fabric structure filled with the extended use biocompatible material.

7. The intervertebral prosthetic disc of claim 8, wherein the second fabric structure includes a plurality of adjacent cylindrical tubes.

8. The intervertebral prosthetic disc of claim 7, wherein the plurality of adjacent cylindrical tubes are interconnected to the extended use biocompatible material to flow there between.

9. The intervertebral prosthetic disc of claim 6, wherein the first fabric structure, the second fabric structure, or a combination thereof comprises poly(L-lactide-co-D, L-lactide) (PLDLLA), polyglycolic acid (PGA), polylactic acid (PLA), collagen, polyethyleneterephthalate (PET), woven titanium, polyetheretherketone (PEEK), carbon, ultra high molecular weight polyethylene (UHMWPE), or a combination thereof.

10. The intervertebral prosthetic disc of claim 6, wherein the extended use biocompatible material is a synthetic polymer, a natural polymer, a bioactive ceramic, carbon nanofibers, or a combination thereof.

11. The intervertebral prosthetic disc of claim 10, wherein the synthetic polymer is a polyurethane material, a polyolefin material, a polyether material, a polyester material, a polycarbonate material, a silicone material, a hydrogel material, or a combination thereof.

12. The intervertebral prosthetic disc of claim 11, wherein the polyolefin material is polypropylene, polyethylene, halogenated polyolefin, flouropolyolefin, or a combination thereof.

13. The intervertebral prosthetic disc of claim 11, wherein the polyether material is polyetherketone (PEK), polyetheretherketone (PEEK), polyetherketoneketone (PEKK), polyaryletherketone (PAEK), or a combination thereof.

14. The intervertebral prosthetic disc of claim 11, wherein the polyester material is polylactide.

15. The intervertebral prosthetic disc of claim 11, wherein the polycarbonate material is tyrosine polycarbonate.

16. The intervertebral prosthetic disc of claim 10, wherein the natural polymer is collagen, gelatin, fibrin, keratin, chitosan, chitin, hyaluronic acid, albumin, silk, elastin, or a combination thereof.

17. The intervertebral prosthetic disc of claim 10, wherein the bioactive ceramic is hydroxyapatite (HA), hydroxyapatite tricalcium phosphate (HATCP), calcium phosphate, calcium sulfate, or a combination thereof.

18. The intervertebral prosthetic disc of claim 5, wherein the first compliant structure, the second compliant structure, or a combination thereof includes a biological factor to promote bone growth.

19. The intervertebral prosthetic disc of claim 18, wherein the biological factor is a bone morphogenetic protein (BMP), a cartilage-derived morphogenetic protein (CDMP), a platelet derived growth factor (PDGF), an insulin-like growth factor (IGF), a LIM mineralization protein, a fibroblast growth factor (FGF), an osteoblast growth factor, stem cells, or a combination thereof.

20. The intervertebral prosthetic disc of claim 19, wherein the stem cells include bone marrow derived stem cells, lipo derived stem cells, or a combination thereof.

21. The intervertebral prosthetic disc of claim 1, further comprising a first tooth extending from the first component.

22. The intervertebral prosthetic disc of claim 21, wherein the first tooth is configured to at least partially protrude through the first compliant structure and engage the first vertebra.

23. The intervertebral prosthetic disc of claim 5, further comprising a second tooth extending from the second component.

24. The intervertebral prosthetic disc of claim 23, wherein the second tooth is configured to at least partially extend through the second compliant structure and engage the second vertebra.

25. An intervertebral prosthetic disc to be installed within an intervertebral space between an inferior vertebra and a superior vertebra, the intervertebral prosthetic disc comprising:
an inferior support plate having an inferior compliant structure attached thereto, wherein the inferior compliant structure is configured to conform to the inferior vertebra; and
a superior support plate having a superior compliant structure attached thereto, wherein the superior compliant structure is configured to conform to the superior vertebra.

26.-32. (canceled)

33. An intervertebral prosthetic disc to be installed within an intervertebral space between an inferior vertebra and a superior vertebra, the intervertebral prosthetic disc comprising:
a superior component, the superior component comprising:
a superior support plate; and
a superior compliant structure affixed to the superior bearing surface;
an inferior component, the inferior component comprising:
an inferior support plate; and
an inferior compliant structure affixed to the inferior bearing surface; and
a nucleus disposed between the superior component and the inferior component, wherein the nucleus is configured to allow relative motion between the superior component and the inferior component.

34.-41. (canceled)
